(19) United States
(12) Patent Application Publication
(10) Pub. No.: US 2022/0186166 A1
(43) Pub. Date: Jun. 16, 2022

(54) SEGMENTED CULTURE DISH AND IMPRINTING SYSTEM

(71) Applicant: Soft Cell Biological Research, Inc., St. George, UT (US)

(72) Inventors: Gregory A. Prince, St. George, UT (US); Wayne A. Provost, Ivins, UT (US)

(73) Assignee: Soft Cell Biological Research, Inc., St. George, UT (US)

(21) Appl. No.: 17/547,955

(22) Filed: Dec. 10, 2021

Related U.S. Application Data
(60) Provisional application No. 63/125,457, filed on Dec. 15, 2020.

(51) Int. Cl. C12M 1/00; C12M 1/22

(52) U.S. Cl. CPC C12M 23/34; C12M 23/38; C12M 23/10

(57) ABSTRACT

A segmented culture dish and imprinting system allows duplicate plates to be produced rapidly and without culture-to-culture overlap. The segmented culture dish and imprinting system includes a dish stamp, a first culture dish, and a second culture dish. The dish stamp may be placed on the first and/or the second culture dishes. The first and the second culture dishes have a key on an inside surface that ensures the orientation of cultures is always preserved, eliminating the need for markings on culture dishes.

### CROSS-REFERENCE TO RELATED APPLICATIONS

This application claims the benefit of U.S. Provisional Application Ser. No. 63/125,457, filed on Dec. 15, 2020, which is incorporated herein by reference.

### TECHNICAL FIELD

The present disclosure relates to a culture dish. More particularly, the present disclosure relates to segmented culture dish with an imprinting apparatus.

### BACKGROUND

A worldwide public health crisis is accelerating the emergence of antibiotic-resistant bacteria and fungi. In spite of this emergence, the number of pharmaceutical companies with antibiotic discovery programs has dropped over the past two decades from eighteen to three. There is a desperate need for new antibiotics to combat the emergence of antibiotic-resistant bacteria and fungi. As an example, there has not been a new class of Gram-negative antibiotics in six decades.

As a part of combating bacteria and fungi, scientists perform tests using culture plates. However, scientists are left with a confusing and frustrating system of using and organizing culture plate screenings when many tests need to be performed. Specifically, during a screening, scientists are generally forced to mark and label the underside of a culture plate with a permanent marker and then smear each segment of the culture plate with a target organism against which activity can be measured.

Additionally, the target organisms placed on the culture plates are not separated by a barrier, such as a wall; rather they are on the same surface of medium, which often leads to overlap of adjacent organisms. Both the marking of the culture plates and the constant overlap of organisms creates frustration and consumes a lot of time, preventing many scientists from efficiently performing tests on organisms.

Additionally, if a user desires to test multiple antibiotics against the same target organisms, they must mark and smear each separate petri dish, which is a very tedious process.

Accordingly, there is a need for a culture plate that segregates organisms, that allows for easy and consistent marking of organisms, and that is easily replicable on additional culture plates. The present disclosure seeks to solve these and other problems.

### SUMMARY OF EXAMPLE EMBODIMENTS

In some embodiments, a segmented culture dish and imprinting system comprises a dish stamp, a first culture dish, and a second culture dish. The dish stamp may be placed on the first and/or the second culture dishes. The dish stamp comprises a top with a handle and a bottom with imprinting segments. The bottom of the dish stamp further comprises channels interposed between the imprinting segments. A keyed portion allows the proper orientation and alignment of the dish stamp when placed on the culture dishes. For example, extending from the top of the dish stamp to the bottom is an orientation channel (one non-limiting example of a “keyed portion”) configured to mate with a key (e.g., a protrusion, indentation, etc.) to orient the dish stamp on the first and the second culture dishes.

In one method of use, a user aligns the keyed portion of the dish stamp with the key of the first culture dish and then presses the bottom of the dish stamp into the culture medium, thereby transferring a portion of each of the plurality of organisms (each plurality segregated by segment walls or ridges) previously placed on the first culture dish to the bottom of the dish stamp. The dish stamp may then be aligned, via the keyed portion once again, with the second culture dish. The dish stamp may then be pressed against the culture medium, transferring at least a portion of each of the plurality of organisms from the bottom of the dish stamp to the culture medium. This process may be repeated any number of times with subsequent culture dishes, as desired. This “stamping” effect allows a user to quickly and easily replicate a plurality of organisms on a plurality of culture dishes. Because the orientation of the dish stamp remains the same in relation to the key of each dish stamp, there is no need to individually mark each culture dish in advance, only the first culture dish. Each segment of the additional culture dishes may be identified by its location in relation to the key, thereby identifying the organism in each segment based upon the first culture dish.

### BRIEF DESCRIPTION OF THE DRAWINGS

FIG. 1 illustrates a top perspective view of a dish stamp of a segmented culture dish and imprinting system;

FIG. 2 illustrates a bottom perspective view of a dish stamp of a segmented culture dish and imprinting system;

FIG. 3 illustrates a top perspective view of a first culture dish of a segmented culture dish and imprinting system;

FIG. 4 illustrates a top perspective view of a second culture dish of a segmented culture dish and imprinting system;

FIG. 5 illustrates a top perspective view of a dish stamp of a segmented culture dish and imprinting system;

FIG. 6 illustrates a bottom perspective view of a dish stamp of a segmented culture dish and imprinting system;

FIG. 7 illustrates a top plan view of a dish stamp of a segmented culture dish and imprinting system;

FIG. 8 illustrates a side elevation view of a dish stamp of a segmented culture dish and imprinting system;

FIG. 9 illustrates a top perspective view of a second culture dish of a segmented culture dish and imprinting system;

FIG. 10 illustrates a top perspective view of a first culture dish of a segmented culture dish and imprinting system;

FIG. 11 illustrates a top plan view of a first culture dish of a segmented culture dish and imprinting system;

FIG. 12 illustrates of a side elevation view of a first culture dish of a segmented culture dish and imprinting system;

FIG. 13 illustrates a top plan view of a second culture dish of a segmented culture dish and imprinting system;

FIG. 14 illustrates a side elevation view of a second culture dish of a segmented culture dish and imprinting system;

FIG. 15 illustrates a top perspective view of a lid of a segmented culture dish and imprinting system;

FIG. 16 illustrates a top plan view of a lid of a segmented culture dish and imprinting system; and

FIG. 17 illustrates a side elevation view of a lid of a segmented culture dish and imprinting system.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As discussed earlier, there is a need for a culture plate that segregates organisms, that allows for easy and consistent marking of organisms, and that is easily replicable on additional culture plates. The segmented culture dish and imprinting system disclosed herein solves these and other problems.

Antibiotic discovery often requires large scale, high throughput of screening. Typically, such screening involves the laborious task of marking and labeling the underside of a culture plate with a permanent marker, and then smearing each segment with a target organism against which activity can be measured. This process is extremely time consuming, especially when large scale screening is required. Further, overlap of adjacent organisms is a constant problem because different types of organisms are placed on the culture without a physical barrier.

The segmented culture dish and imprinting system described herein allows duplicate plates to be produced rapidly and without culture-to-culture overlap. The system comprises a first culture dish, a second culture dish, and a dish stamp. The first and the second culture dishes comprise a key (e.g., an indentation, guide ridge, or other protrusion) that ensures the orientation of cultures is always preserved, eliminating the need for markings on culture dishes.

Furthermore, the first culture dish comprises segment walls to separate the organisms, keeping them from overlapping. To transfer organisms from the first culture dish, a user positions a dish stamp having imprinting segments that contact the organisms located at culture segments between the segment walls on the first culture dish. This allows for transfer of microorganisms from the surface of the culture medium of the first culture dish to an uninoculated semi-solid culture medium on the second culture dish. Orientation of the organisms is maintained via a key on the second culture dish. It will be appreciated that the segmented culture dish and imprinting system described herein allows a user to easily orient organisms and prevent overlap, facilitating high-throughput evaluation of microorganisms to screen for potential anti-microbial activity.

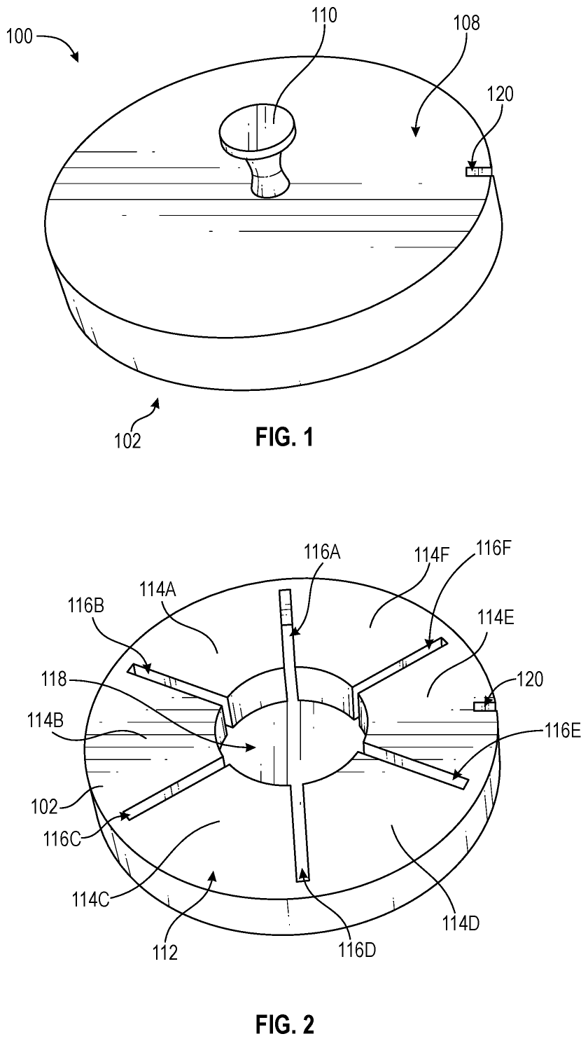

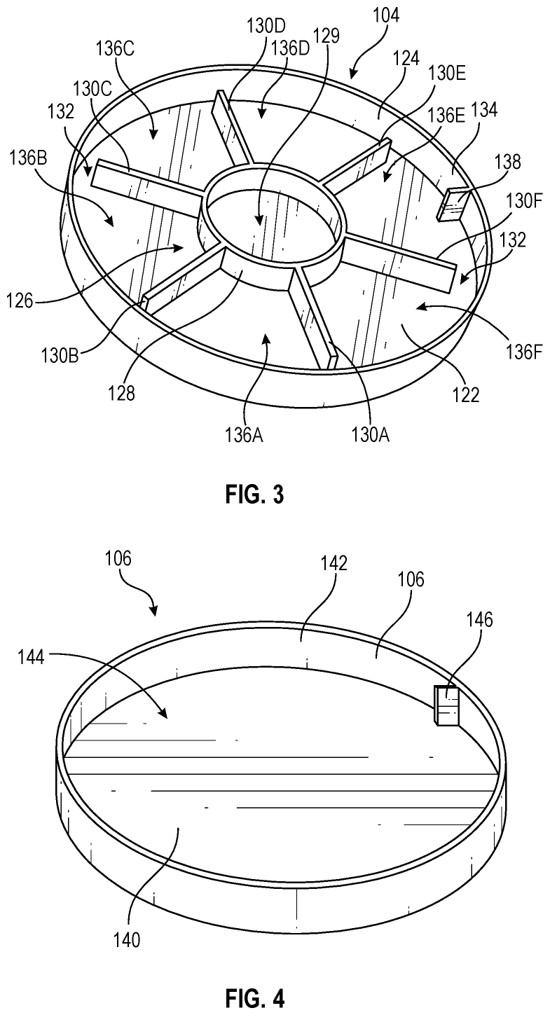

Referring now to FIGS. 1-5, in one embodiment, a segmented culture dish and imprinting system 100 comprises a dish stamp 102, a first culture dish 104 (FIG. 3), and a second culture dish 106 (FIG. 4). The dish stamp 102 comprises a top 108 with a handle 110 and a bottom 112 with imprinting segments 114A-F segregated by channels 116A-F. The imprinting segments 114A-F may be used to transfer organisms from the first culture dish 104 to the second culture dish 106. The bottom 112 of the dish stamp 102 may further comprise a void 118. The void 118 may be used to ensure that no organisms are transferred from the first culture dish 104 to the second culture dish 106 where the void is located, allowing a user to place differing antibiotics (or other test materials) in that position of the second culture dish. A keyed portion 120 (e.g., orientation channel) may extend vertically from the top 108 to the bottom 112 of the dish stamp 102. The keyed portion 120 is configured to receive a key 138, 146 of the first and second culture dishes 104, 106, respectively. For example, the key 138, 146 may be a protrusion that extends vertically from the top to the bottom of the culture dish, thereby ensuring orientation of the dish stamp 102 as it is pressed thereon.

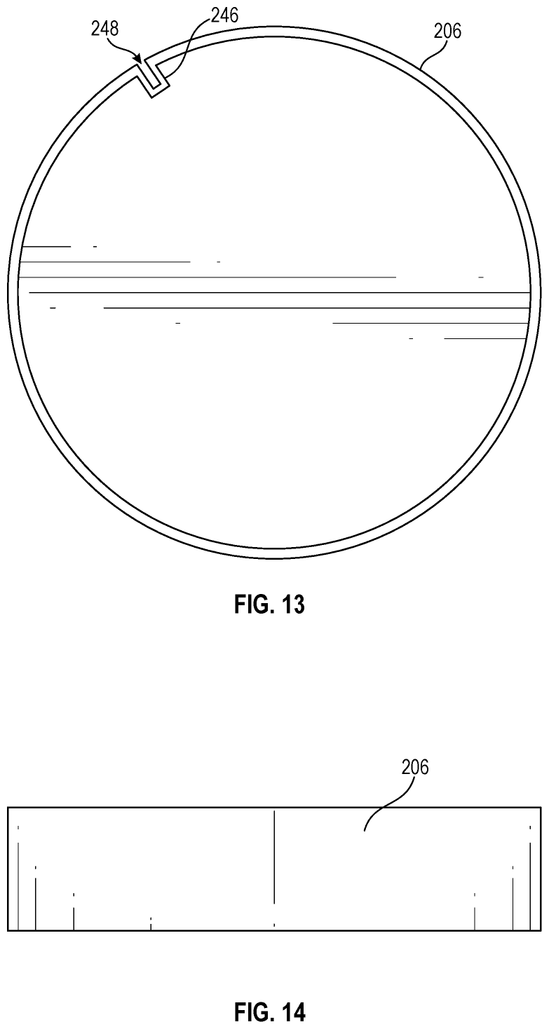

Referring to FIG. 3, the first culture dish 104 comprises a base 122 with an outer, vertical wall 124. The first culture dish 104 may be a round dish; however, it will be appreciated that other shapes of culture dishes may be envisioned, such as square, rectangular, hexagonal, or other geometric shapes. In a center and on a top surface 126, the base 122 comprises an inner wall 128, which may be circular, creating a void 129 therein and preserving a central space (e.g., free from bacteria or other organisms) into which the candidate antimicrobial (or other test substance) culture may be applied on the second culture dish 106. While the inner wall 128 is illustrated as being in a circular shape, it will be understood that the inner wall 128 is not limited to being circular and may be other shapes so long as it creates a void so as to prohibit culture medium from entering the void. Additionally, the void 129 need not be negative space, but may also be solid, as shown in FIGS. 13-14.

Radiating from the inner wall 128 towards the outer wall 124 are segment walls 130A-F, which may be of the same height as the inner wall 128, or any other height. Each segment wall 130A-F corresponds to a channel 116A-F of the dish stamp 102. The segment walls 130A-F form culture segments 136A-F that correspond to the imprinting segments 114A-F. While six segments 114A-F, 136A-F are shown, more or less segments may be used without departing herefrom.

Each of the segment walls 130A-F extend from the inner wall 128 and toward the outer wall 124, but do not contact the outer wall 124, thereby leaving a culture medium gap 132 between each segment wall 130A-F and an inside surface 134 of the outer wall 124. The culture medium gap 132 allows semi-soft media, when poured into the first culture dish 104, to self-level in all culture segments 136A-F prior to becoming semi-solid. It should be noted that this process saves time and ensures the culture medium is the same height in all culture segments 136A-F. The channels 116A-F on the dish stamp 102 are complementary sized and shaped so as to receive the corresponding segment walls 130A-F.

Each of the culture segments 136A-F may receive a different organism (e.g., bacteria). It will be appreciated that the culture segments 136A-F are segregated from each other by the segment walls 130A-F so as to prevent overlapping of organisms, overcoming the problems in the prior art. Along the vertical outer wall 124 of the first culture dish 104, at a point bisecting one of the segments 136A-F, is a key 138 (e.g., protrusion) of at least the same height as the segment walls 130A-F which mates with the keyed portion 120 of the dish stamp 102 so as to assist a user in orienting and placing the dish stamp 102 on the first culture dish 104.

Referring to FIG. 4, a second culture dish 106, in some embodiments, comprises a base 140 with a vertical peripheral wall 142 forming a central void 144 to receive culture medium. The second culture dish 106 comprises a key 146 (e.g., protrusion), similar to the key 138, to orient and receive the dish stamp 102. Accordingly, the culture medium in the void 144 will receive the bacteria from each imprinting segment 114A-F, creating a contaminated segment, thereby allowing each imprinted bacteria to multiply separate from each other. Due to the void 129 in the first culture dish 104 and the void 118 in the dish stamp 102, the portion of the culture medium of the second dish 106 is left uncontaminated. As a result, a user may place an antibiotic candidate in the uncontaminated portion to view how it affects each contaminated segment. A user may utilize more than two dishes, with each subsequent dish having the same form as the second culture dish 106. As a result, numerous antibiotics may be trialed with ease by simply repeating the stamping process—placing the dish stamp 102 on the first culture dish 104 and then placing the dish stamp 102 on a subsequent culture dish having the same configuration as the second culture dish 106.

To use the segmented culture dish and imprinting system 100, a user pours culture medium into a segment 136A-F of the first culture dish 104, which proceeds to fill each culture segment 136A-F via the culture medium gaps 132, leaving the inner void 129 within the inner wall 128 free of medium. These culture medium gaps 132 allow the culture medium to self-level prior to solidifying. Once the medium solidifies (e.g., agar becomes semi-solid), organisms may be introduced to each culture segment 136A-F separated by the segment walls 130A-F. After growth of the organisms in the culture segments 136A-F, the user may position the dish stamp 102 on the first culture dish 104 by aligning the keyed portion 120 (e.g., channel) with the key 138 (e.g., protrusion) and inserting the segment walls 130A-F into the corresponding channels 116A-F, thereby allowing the imprinting segments 114A-F to be pressed against the surface of the semi-solid medium in which microorganisms are cultured.

The user may then remove the dish stamp 102 and align the keyed portion 120 with the second key 146 of the second culture dish 106, effectively transferring the organisms from the first culture dish 104 to the uncontaminated medium of the second culture dish 106. It will be appreciated that the orientation of the organisms remain consistent from dish to dish due to the keyed portion 120 when transferring organisms from the first culture dish 104 to the second culture dish 106, removing the need to mark on the bottom of the second culture dish 106 and subsequent culture dishes where an organism is located. It will further be appreciated that the system 100 is not limited to a single keyed portion 120 and keys 138, 146, but may have a plurality of keys on a single dish so long as it allows only a single orientation for placement. Due to the fact that an organism is not placed in the void 129 within the inner circle 128, when the organisms are transferred to the second culture dish 106, the center of the culture medium of the second culture dish 106 will be lacking an organism and ready to receive an antibiotic.

The dish stamp 102 may then be repeatedly used to transfer cultures from the first culture dish 104 to any number of culture dishes configured as the second culture dish 106. This allows for rapid transference of organisms to many culture dishes, allowing a user to quickly test the efficacy of many antibiotics and many organisms. A lid may then be placed on each culture dish 106 to prohibit additional contamination of the medium. As discussed later herein, the lid may also have a keyed portion, allowing a user to write on the outer surface of the lid, if desired, without fear of the orientation of the lid changing. Accordingly, the segmented culture dish and imprinting system 100 overcomes the problems in the art described earlier herein.

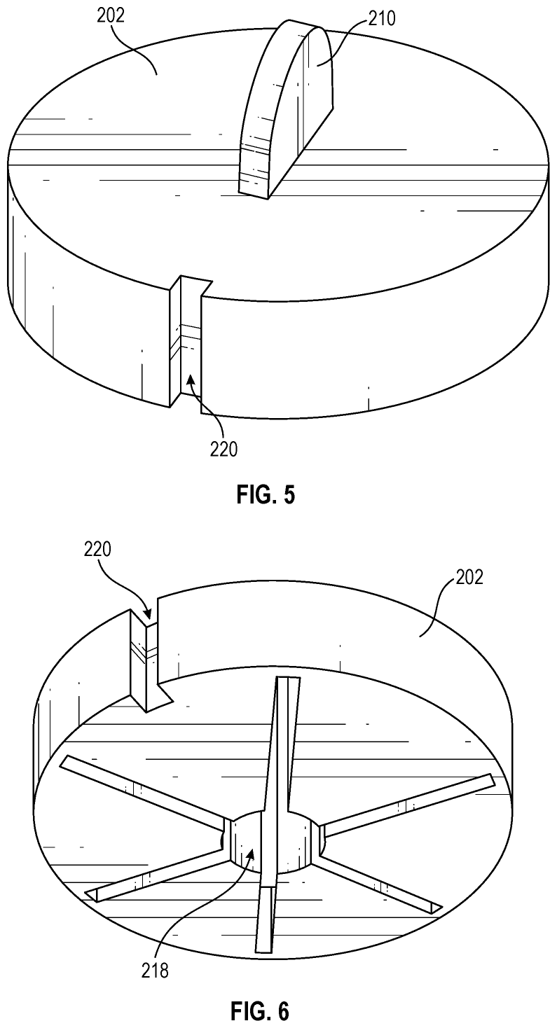

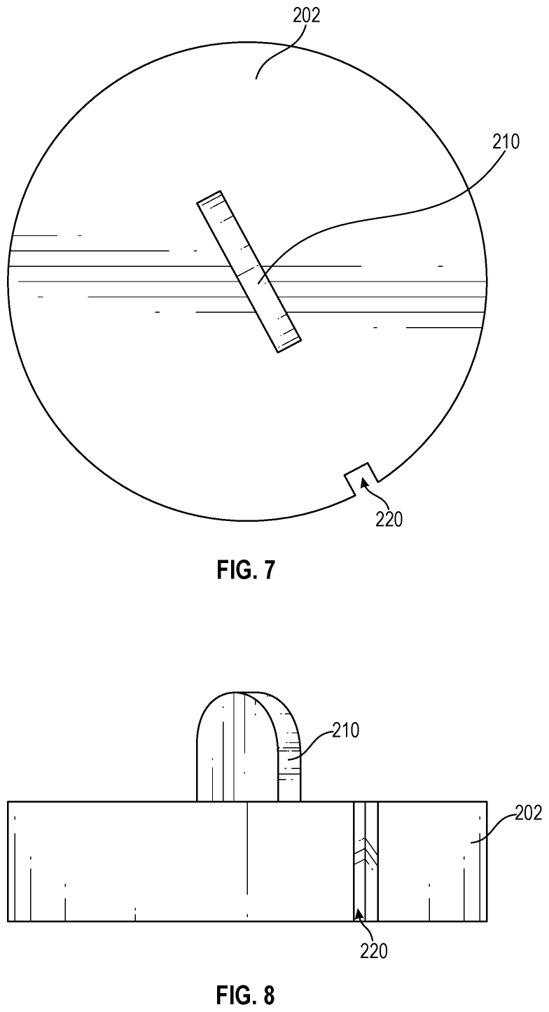

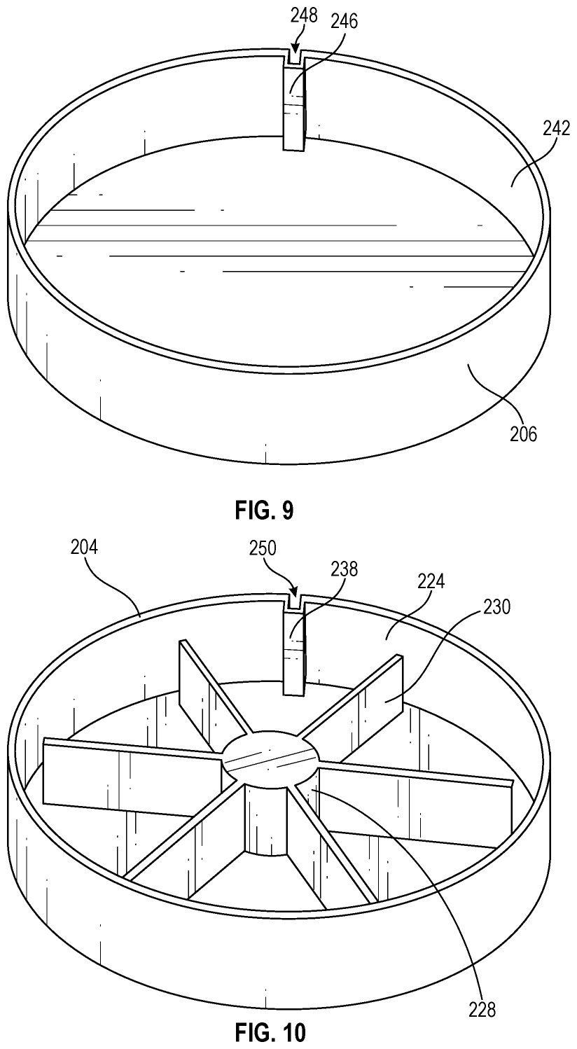

In some embodiments, as shown in FIGS. 5-8, a dish stamp 202 comprises a handle 210 thereon. The handle 210 may be part of the manufacturing mold, thereby creating a dish stamp 202 and a handle 210 as a single integrated unit, reducing assembly and thereby decreasing cost. In some embodiments, the handle 110, 210 may be coupled to the dish stamp 102, 202 via a screw, bolt and nut, or other coupling means. Alternatively, in some embodiments, the dish stamp 102, 202 may not have a handle, but may have a lip or raised edge that a user can grip to remove the dish stamp 102, 202. Additionally, the keyed portion 220 may be a channel or aperture in the sidewall (e.g., an indentation forming a slot) of the dish stamp 202, which may be formed in the same mold, as well as the void 218 and accompanying channels. Referring to FIGS. 9 and 13-14, in some embodiments, the key 246 of the second culture dish 206 may be a protrusion formed in the vertical peripheral wall 242, forming an aperture 248 in the outer circumference of the vertical peripheral wall 242 (e.g., an indentation forming a slot on the outside and a protrusion on the inside). It will be appreciated that the key 246 is sized so as to be received in the keyed portion 220 (e.g., tongue and groove relationship).

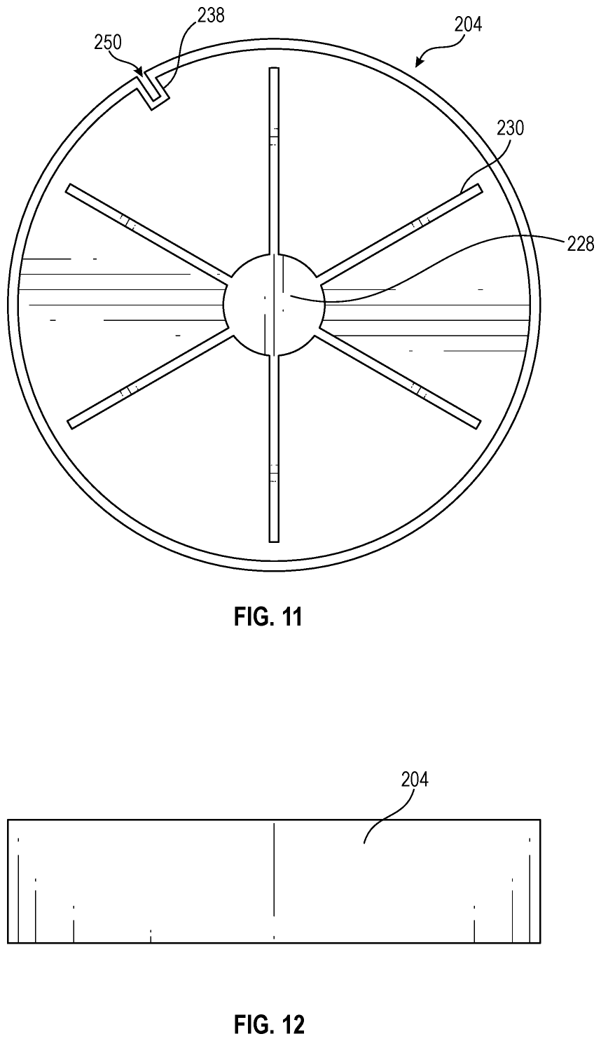

Referring to FIGS. 10-12, the first culture dish 204 likewise comprises a key 238 that is formed in the vertical wall 224, forming an aperture 250 in the outer circumference of the vertical wall 224. The key 238 is sized so as to be received in the keyed portion 220 (e.g., tongue and groove relationship). In some embodiments, as shown, the inner wall 228 may be solid (instead of forming a void 129) with one or more segment walls 230 extending radially therefrom. The solid inner wall 228 is receivable within the void 218 of the dish stamp 202.

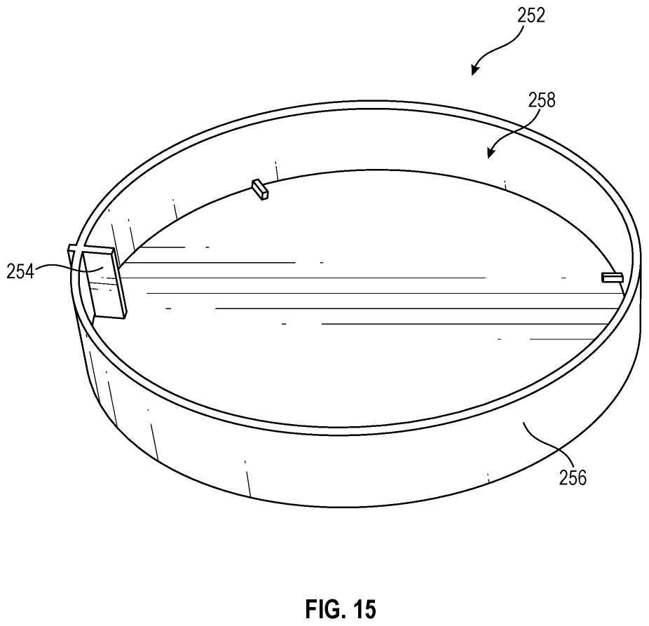

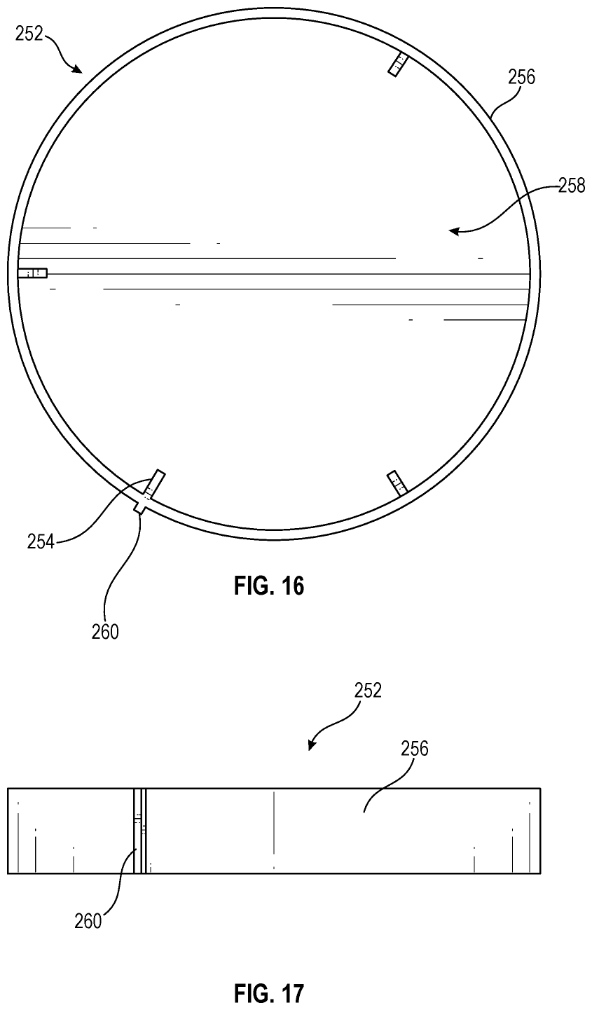

Referring now to FIGS. 15-17, a lid 252 comprises a lid key 254 extending inwardly from the vertical wall 256. The vertical wall encloses a void 258 that is configured to receive the second culture dish 206. In other words, the vertical wall has an inner diameter greater than the outer diameter of the second culture dish 206, such that the second culture dish 206 is enclosed therein. The key 254 (e.g., protrusion) slides into the aperture 248 of the second culture dish 206, thereby maintaining consistent positioning. As a result, a user may write on the lid 252, if desired, without fearing that the lid 252 will rotate. In some embodiments, a protrusion 260 may extend outwardly to indicate the position of they key 254, allowing a user to more easily match up the position of the lid 252 with the second culture dish 206.

Accordingly, as appreciated from the foregoing disclosure, the segmented culture dish and imprinting system disclosed herein solves the need for a culture plate that segregates organisms, that allows for easy and consistent marking of organisms, and that is easily replicable on additional culture plates.

It will also be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A segmented culture dish and imprinting system, comprising:
a dish stamp comprising a plurality of imprinting segments and a keyed portion;
a first culture dish comprising a plurality of culture segments and a key; and
a second culture dish comprising a key;
wherein the keyed portion is configured to receive the key of the first culture dish
and the key of the second culture dish.

2. The segmented culture dish and imprinting system of claim 1, wherein the dish stamp comprises a handle.

3. The segmented culture dish and imprinting system of claim 1, wherein each imprinting segment of the dish stamp is separated by a channel.

4. The segmented culture dish and imprinting system of claim 1, wherein the dish stamp further comprises a void.

5. The segmented culture dish and imprinting system of claim 1, wherein the culture segments of the first culture dish are separated by segment walls.

6. The segmented culture dish and imprinting system of claim 1, wherein the first culture dish further comprises an inner wall.

7. The segmented culture dish and imprinting system of claim 6, further comprising segmented walls extending radially outward from the inner wall to separate the culture segments.

8. The segmented culture dish and imprinting system of claim 7, further comprising a culture medium gap at an end of each segmented wall.

9. The segmented culture dish and imprinting system of claim 1, further comprising a lid, the lid comprising a key.

10. A segmented culture dish and imprinting system, comprising:
a dish stamp comprising:
a plurality of imprinting segments, each imprinting segment separated by a
channel,
a center void,
a keyed portion, and
a handle;
a first culture dish comprising:
a center wall,
a plurality of wall segments extending radially from the center wall, the wall segments forming a plurality of culture segments,
a culture medium gap between an end of each wall segment and an outer vertical wall,
and a key;
a second culture dish comprising a key; and
a lid comprising a key;
wherein the keyed portion of the dish stamp is configured to receive the key of the first culture dish, the key of the second culture dish, and the key of the lid.

11. The segmented culture dish and imprinting system of claim 10, wherein the keyed portion is an aperture.

12. The segmented culture dish and imprinting system of claim 10, wherein the key of the first dish culture is a protrusion.

13. A method of using a segmented culture dish and imprinting system, the method comprising:
pouring liquid medium into a first culture dish, the first liquid dish comprising at least one segment wall dividing at least two culture segments, the liquid medium self-leveling via at least one culture medium gap between the segment wall and a peripheral vertical wall;
once the liquid medium has solidified, applying a first organism to a first culture segment of the at least two culture segments and applying a second organism to a second culture second segment of the at least two culture segments;
pressing a dish stamp onto the solidified culture medium to transfer the first and second organisms to the dish stamp; and
pressing the dish stamp onto a second culture dish, the second culture dish comprising solidified culture medium, transferring the first and second organisms to the solidified culture medium of the second culture dish.

14. The method of claim 13, further comprising aligning a keyed portion of the dish stamp with a key of the first culture dish and a key of the second culture dish.

15. The method of claim 13, further comprising aligning a keyed portion of a lid with a key of the second culture dish.
